SEQUENCE LISTING (4 sequences)

SEQ ID NO: 1 - protein, 19 aa, Streptoverticillium cinnamoneum
  MISC_FEATURE (1)..(1): F-18 radioisotope attached via a linker group
CRQSCSFGPPTFVCXGNTK

SEQ ID NO: 2 - protein, 19 aa, Streptoverticillium cinnamoneum
  THIOETH (1)..(18)
  MISC_FEATURE (1)..(1): F-18 radioisotope attached via a linker group
CKQSCSFGPPTFVCXGNTK

SEQ ID NO: 3 - protein, 19 aa, Streptoverticillium cinnamoneum
  THIOETH (1)..(18)
  MISC_FEATURE (1)..(1): F-18 radioisotope attached via a linker group
CKQSCSFGPPTFVCXGNTK

SEQ ID NO: 4 - protein, 19 aa, Streptoverticillium cinnamoneum
  THIOETH (1)..(18)
  MISC_FEATURE (2)..(2): F-18 radioisotope attached via a linker group
CKQSCSFGPPTFVCXGNTK

(19) United States
(12) Patent Application Publication
(10) Pub. No.: US 2013/0251632 A1
(43) Pub. Date: Sep. 26, 2013

(54) APOPTOSIS PET IMAGING AGENTS

(71) Applicants: Duncan Hiscock, Amersham (GB); Bente Elizabeth Arbo, Oslo (NO); Graeme Walter Mcrobbie, Amersham (GB); Bard Indrevoll, Oslo (NO); Rajiv Bhalla, Amersham (GB)

(72) Inventors: Duncan Hiscock, Amersham (GB); Bente Elizabeth Arbo, Oslo (NO); Graeme Walter Mcrobbie, Amersham (GB); Bard Indrevoll, Oslo (NO); Rajiv Bhalla, Amersham (GB)

(73) Assignee: GE HEALTHCARE LIMITED, BUCKINGHAMSHIRE (GB)

(21) Appl. No.: 13/991,260

(22) PCT Filed: Dec. 1, 2011
(86) PCT No.: PCT/EP2011/071484

§ 371 Date: Jun. 3, 2013

(30) Foreign Application Priority Data
Dec. 1, 2010 (GB) 1020314.9

(51) Int. Cl. A61K 51/08

(52) U.S. Cl. CPC A61K 51/088
USPC 424/1.69; 530/326

(57) ABSTRACT

The present invention relates to radiopharmaceutical imaging in vivo of apoptosis and other forms of cell death. The invention provides PET imaging agents which target apoptotic cells via selective binding to the aminophospholipid phosphatidylethanolamine (PE), which is exposed on the surface of apoptotic cells. Also provided are pharmaceutical compositions, kits and methods of in vivo imaging.

### FIELD OF THE INVENTION

The present invention relates to radiopharmaceutical imaging in vivo of apoptosis and other forms of cell death. The invention provides PET imaging agents which target apoptotic cells via selective binding to the aminophospholipid phosphatidylethanolamine (PE), which is exposed on the surface of apoptotic cells. Also provided are pharmaceutical compositions, kits and methods of in vivo imaging.

### BACKGROUND TO THE INVENTION

Apoptosis or programmed cell death (PCD) is the most prevalent cell death pathway and proceeds via a highly regulated, energy-conserved mechanism. In the healthy state, apoptosis plays a pivotal role in controlling cell growth, regulating cell number, facilitating morphogenesis, and removing harmful or abnormal cells. Dysregulation of the PCD process has been implicated in a number of disease states, including those associated with the inhibition of apoptosis, such as cancer and autoimmune disorders, and those associated with hyperactive apoptosis, including neurodegenerative diseases, haematologic diseases, AIDS, ischaemia and allograft rejection. The visualization and quantitation of apoptosis is therefore useful in the diagnosis of such apoptosis-related pathophysiology.

Therapeutic treatments for these diseases aim to restore balanced apoptosis, either by stimulating or inhibiting the PCD process as appropriate. Non-invasive imaging of apoptosis in cells and tissue in vivo is therefore of immense value for early assessment of a response to therapeutic intervention, and can provide new insight into devastating pathological processes. Of particular interest is early monitoring of the efficacy of cancer therapy to ensure that malignant growth is controlled before the condition becomes terminal.

There has consequently been great interest in developing imaging agents for apoptosis [see eg. Zeng et al, Anti-cancer Agent Med. Chem., 9(9), 986-995 (2009); Zhao, ibid, 9(9), 1018-1023 (2009) and M. De Saint-Hubert et al, Methods, 48, 178-187 (2009)]. Of the probes available for imaging cell death, radiolabelled Annexin V has received the most attention. Annexin V binds only to negatively charged phospholipids, which renders it unable to distinguish between apoptosis and necrosis.

The lanthionine-containing antibiotic peptides (“lantibiotics”) duramycin and cinnamycin are two closely related 19-mer peptides with a compact tetracyclic structure [Zhao, Amino Acids, DOI 10.1007/s00726-009-0386-9, Springer-Verlag (2009), and references cited therein]. They are crosslinked via four covalent, intramolecular bridges, and differ by only a single amino acid residue at position 2. The structures of duramycin and cinnamycin are shown schematically below, where the numbering refers to the position of the linked amino acid residues in the 19-mer sequence:

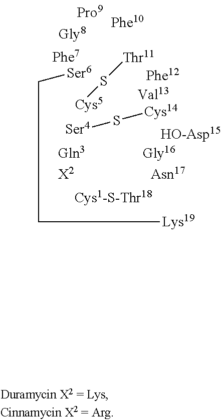

Programmed cell death or apoptosis is an intracellular, energy-dependent self-destruction of the cell. The redistribution of phospholipids across the bilayer of the cell plasma membrane is an important marker for apoptosis. Thus, in viable cells, the aminophospholipids phosphatidylethanolamine (PE) and phosphatidylserine (PS) are predominantly constituents of the inner leaflet of the cell plasma membrane. In apoptotic cells, there is a synchronised externalization of PE and PS.

Both duramycin and cinnamycin bind to the neutral aminophospholipid PE with similar specificity and high affinity, by forming a hydrophobic pocket that fits around the PE head-group. The binding is stabilised by ionic interaction between the β-hydroxyaspartic acid residue (HO-Asp¹⁵) and the ethanolamine group. Modifications to this residue are known to inactivate duramycin [Zhao et al, J. Nucl. Med, 49, 1345-1352 (2008)]. Zhao [Amino Acids, DOI 10.1007/s00726-009-0386-9, Springer-Verlag (2009)] cites earlier work by Wakamatsu et al [Biochemistry, 29, 113-188 (1990)], where NMR studies show that none of the ¹H NMR resonances of the 5 terminal amino acids of cinnamycin are shifted on binding to PE—suggesting that they are not involved in interactions with PE.

US 2004/0147440 A1 (University of Texas System) describes labelled anti-aminophospholipid antibodies, which can be used to detect pre-apoptotic or apoptotic cells, or in cancer imaging. Also provided are conjugates of duramycin with biotin, proteins or anti-viral drugs for cancer therapy.

WO 2006/055855 discloses methods of imaging apoptosis using a radiolabelled compound which comprises a phosphatidylserine-binding C2 domain of a protein.

WO 2009/114549 discloses a radiopharmaceutical made by a process comprising:
(i) providing a polypeptide having at least 70% sequence similarity with CKQSCSFGPFTFVCDGNTK,
   wherein the polypeptide comprises a thioether bond between amino acids residues 1-18, 4-14, and 5-11, and an amide bond between amino acids residues 6-19, and, wherein one or more distal moieties of structure

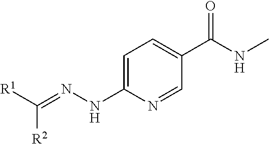

are covalently bound to the amino acid at position 1, position 2, or, positions 1 and 2 of the polypeptide, and wherein R¹ and R² are each independently a straight or branched, saturated or unsaturated C₁₋₄ alkyl; and
(ii) chelating one or more of the distal moieties with ^{99m}Tc^{x}, (^{99m}Tc═O)³⁺, (^{99m}Tc≡N)²⁺, (O═^{99m}Tc═O)⁺ or [^{99m}Tc(CO)₃]⁺, wherein x is a redox or oxidation state selected from the group consisting of +7, +6, +5, +4, +3, +2, +1, 0 and −1, or, a salt, solvate or hydrate thereof.

The ‘distal moiety’ of WO 2009/114549 is a complexing agent for the radioisotope ^{99m}Tc, which is based on hydrazinonicotinamide (commonly abbreviated “HYNIC”). HYNIC is well known in the literature [see e.g. Banerjee et al, Nucl. Med. Biol, 32, 1-20 (2005)], and is a preferred method of labelling peptides and proteins with ^{99m}Tc [R. Alberto, Chapter 2, pages 19-40 in IAEA Radioisotopes and Radiopharmaceuticals Series 1: “Technetium-99m Radiopharmaceuticals Status and Trends” (2009)].

WO 2009/114549 discloses specifically ^{99m}Tc-HYNIC-duramycin, and suggests that the radiopharmaceuticals taught therein are useful for imaging apoptosis and/or necrosis, atherosclerotic plaque or acute myocardial infarct.

Zhao et al [J. Nucl. Med, 49, 1345-1352 (2008)] disclose the preparation of ^{99m}Tc-HYNIC-duramycin. Zhao et al note that duramycin has 2 amine groups available for conjugation to HYNIC: at the N-terminus (Cys1 residue), and the epsilon-amine side chain of the Lys2 residue. They purified the HYNIC-duramycin conjugate by HPLC to remove the bis-HYNIC-functionalised duramycin, prior to radiolabelling with ^{99m}Tc. Zhao et al acknowledge that the ^{99m}Tc-labelled mono-HYNIC-duramycin conjugates studied are probably in the form of a mixture of isomers.

Whilst HYNIC forms stable ^{99m}Tc complexes, it requires additional co-ligands to complete the coordination sphere of the technetium metal complex. The HYNIC may function as a monodentate ligand or as a bidentate chelator depending on the nature of the amino acid side chain functional groups in the vicinity [King et al, Dalton Trans., 4998-5007 (2007); Meszaros et al [Inorg. Chim. Acta, 363, 1059-1069 (2010)]. Thus, depending on the environment, HYNIC forms metal complexes having 1- or 2-metal donor atoms. Meszaros et al note that the nature of the co-ligands used with HYNIC can have a significant effect on the behaviour of the system, and state that none of the co-ligands is ideal.

### THE PRESENT INVENTION

The present invention provides radiopharmaceutical imaging agents, particularly for imaging disease states of the mammalian body where abnormal apoptosis is involved. The imaging agents comprise an ¹⁸F-radiolabelled lantibiotic peptide.

The invention provides radiotracers which form reproducibly, in high radiochemical purity (RCP). The present inventors have also established that attachment of the radiolabel complex at the N-terminus (Cys^{a} residue) of the lantibiotic peptide of Formula II herein is strongly preferred, since attachment at even the amino acid adjacent to the N-terminus (Xaa of Formula II) has a deleterious effect on binding to phosphatidylethanolamine. This effect was not recognized previously in the prior art, and hence the degree of impact on binding affinity is believed novel.

The ¹⁸F-labelled imaging agents of the present invention are suitable for PET (Positron Emission Tomography), which has the advantage over the imaging agents of the prior art of more facile quantitation of the image.

### DETAILED DESCRIPTION OF THE INVENTION

In a first aspect, the present invention provides an imaging agent which comprises a compound of Formula I:

Z¹-(L)ₙ-[LBP]-Z² (I)
wherein:
LBP is a lantibiotic peptide of Formula II:

Cys^{a}-Xaa-Gln-Ser^{b}-Cys^{c}-Ser^{d}-Phe-Gly-Pro-Phe-Thr^{c}-Phe-Val-Cys^{b}-(HO-Asp)-Gly-Asn-Thr^{a}-Lys^{d} (II)
Xaa is Arg or Lys;
Cys^{a}-Thr^{a}, Ser^{b}-Cys^{b} and Cys^{c}-Thr^{c} are covalently linked via thioether bonds;
Ser^{d}-Lys^{d} are covalently linked via a lysinoalanine bond;
HO-Asp is β-hydroxyaspartic acid;
Z¹-(L)ₙ- is attached to Cys^{a} and optionally also to Xaa of LBP when Xaa is Lys, wherein Z¹ is either ¹⁸F or ¹⁸F coordinated to the metal of a metal complex;
Z² is attached to the C-terminus of LBP and is OH or OB^{c},
   where B^{c} is a biocompatible cation;
L is a synthetic linker group of formula -(A)ₘ- wherein each A is independently —CR₂—, —CR═CR—, —C≡C—, —CR₂CO₂—, —CO₂CR₂—, —NRCO—, —CONR—, —CR═N—O—, —NR(C═O)NR—, —NR(C═S)NR—, —SO₂NR—, —NRSO₂—CR₂OCR₂—, —CR₂SCR₂—, —CR₂NRCR₂—, a C₄₋₈ cycloheteroalkylene group, a C₄₋₈ cycloalkylene group, —Ar—, —NR—Ar—, —O—Ar—, —Ar—(CO)—, an amino acid, a sugar or a monodisperse polyethyleneglycol (PEG) building block, wherein each Ar is independently a C₅₋₁₂ arylene group, or a C₃₋₁₂ heteroarylene group, and wherein each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
   n is an integer of value 0 or 1.

The imaging agents of the present invention are ¹⁸F-labelled lantibiotic peptides. By the term “¹⁸F-radiolabelled” or “¹⁸F-labelled” is meant that the lantibiotic peptide has covalently conjugated thereto the radioisotope ¹⁸F. The ¹⁸F is suitably attached via a C—F fluoroalkyl or fluoroaryl bond, since such bonds are relatively stable in vivo, and hence confer resistance to metabolic cleavage of the ¹⁸F radiolabel from the peptide.

By the term “imaging agent” is meant a compound suitable for imaging the mammalian body. Preferably, the mammal is an intact mammalian body in vivo, and is more preferably a human subject. Preferably, the imaging agent can be administered to the mammalian body in a minimally invasive manner, i.e. without a substantial health risk to the mammalian subject when carried out under professional medical expertise. Such minimally invasive administration is preferably intravenous administration into a peripheral vein of said subject, without the need for local or general anaesthetic. The imaging agents of the first aspect are particularly suitable for imaging apoptosis and other forms of cell death, as is described in the sixth aspect (below).

The term “in vivo imaging” as used herein refers to those techniques that non-invasively produce images of all or part of an internal aspect of a mammalian subject. A preferred imaging technique of the present invention is positron emission tomography (PET).

By the term “metal complex” is meant a coordination complex of a non-radioactive metal. Preferred such complexes comprise a chelating agent. Suitable non-radioactive metals of the invention include aluminium, gallium or indium.

By the term “amino acid” is meant an L- or D-amino acid, amino acid analogue (eg. naphthylalanine) or amino acid mimetic which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure.

“By the term “monodisperse polyethyleneglycol (PEG) building block” is meant PEG biomodifiers of Formula IA or IB:

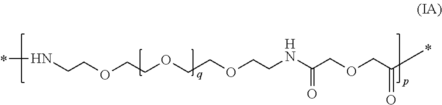

17-amino-5-oxo-6-aza-3,9,12,15-tetraoxaheptadecanoic acid of Formula IA
   wherein q is an integer from 1 to 15 and p is an integer from 1 to 10. Alternatively, a PEG-like structure based on a propionic acid derivative of Formula IB can be used:

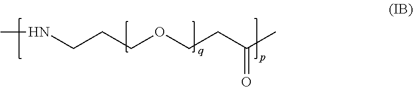

where p and q are as defined for Formula IA and
   In Formula IB, p is preferably 1 or 2, and q is preferably 1 to 12.

By the term “peptide” is meant a compound comprising two or more amino acids, as defined above, linked by a peptide bond (i.e. an amide bond linking the amine of one amino acid to the carboxyl of another).

The term “lantibiotic peptide” refers to a peptide containing at least one lanthionine bond. “Lanthionine” has its conventional meaning, and refers to the sulfide analogue of cystine, having the chemical structure shown:

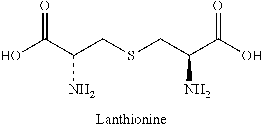 [SMILES: N[C@@H](CSC[C@H](N)C(=O)O)C(=O)O]

By the term “covalently linked via thioether bonds” is meant that the thiol functional group of the relevant Cys residue is linked as a thioether bond to the Ser or Thr residue shown via dehydration of the hydroxyl functional group of the Ser or Thr residue, to give lanthionine or methyllanthionine linkages. Such linkages are described by Willey et al [Ann. Rev. Microbiol., 61, 477-501 (2007)].

By the term “lysinoalanine bond” is meant that the epsilon amine group of the Lys residue is linked as an amine bond to the Ser residue shown via dehydration of the hydroxyl functional group of the Ser giving a —(CH₂)—NH—(CH₂)₄— linkage joining the two alpha-carbon atoms of the amino acid residues.

When Z¹ is attached to Cys^{a}, it is attached to the N-terminus of the LBP peptide. When Z¹ is also attached to Xaa, that means that Xaa is Lys, and Z¹ is attached to the epsilon amino group of the Lys residue.

The Z² group substitutes the carbonyl group of the last amino acid residue of the LBP—i.e. the carboxy terminus. Thus, when Z² is OH, the carboxy terminus of the LBP terminates in the free CO₂H group of the last amino acid residue, and when Z² is OB^{c} that terminal carboxy group is ionised as a CO₂B^{c} group.

By the term “biocompatible cation” (B^{c}) is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

### Preferred Embodiments

In the imaging agent of the first aspect, Z¹ is preferably attached only to Cys^{a} of LBP. When Xaa is Arg, that means that Z¹ is attached to the LBP N-terminus, at the free amino group of the Cys^{a} residue. When Xaa is Lys, that means that steps are taken to either:
(i) selectively functionalise the LBP peptide at the Cys^{a} residue in preference to the epsilon amine group of the Xaa residue; or
(ii) a composition comprising LBP functionalized with Z¹ either at Cys^{a} or at Xaa is prepared, then the Xaa-functionalised species is removed.

In the imaging agent of the first aspect, Xaa is preferably Arg. Z² is preferably OH or OB^{c}.

In Formula I, n is preferably 1, i.e. the linker group (L) is present. When Z¹ is ¹⁸F, preferred radiofluorinated substituents ¹⁸F-(L)ₙ- are of Formula X, wherein -(L)ₙ- is chosen to be —X¹-(A)ₓ-:

¹⁸F—X¹-(A)ₓ- (X)

where: x is an integer of value 0 to 5;
X¹ is chosen from —Ar—, —Ar—NR—, —Ar—O—, —Ar—(CO)— or —Si(R^{a})₂—;
   wherein A, Ar and R are as defined for the L group (above) and each R^{a} is independently C₁₋₉ alkyl.

The Ar group of Ar¹ is preferably a C₁₋₆ aryl group, wherein the ¹⁸F radiolabel is covalently bonded to said aryl group. Ar¹ preferably comprises a phenyl ring or a heterocyclic ring chosen from a triazole, isoxazole or pyridine ring.

When X¹ is —Si(R^{a})₂—, R^{a} can be linear or branched or combinations thereof. R^{a} is preferably branched, and is preferably —C(CH₃)₃. More preferably, both R^{a} groups are —C(CH₃)₃.

In one embodiment, most preferred substituents of Formula X arise from either N-acylation of the N^{α}-amino group of the Cys residue or the N^{ε}-amino group of Lys in LBP with a fluorinated active ester, or condensation of an amino-oxy derivative of the Cys or Lys amine residue with a radiofluorinated benzaldehyde, and comprise the following structural elements:

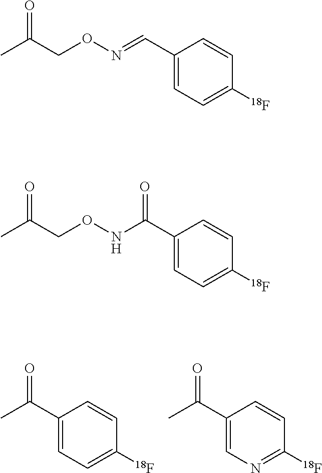

In another embodiment, most preferred substituents of Formula X comprise triazole or isoxazole rings, which arise from click cyclisation:

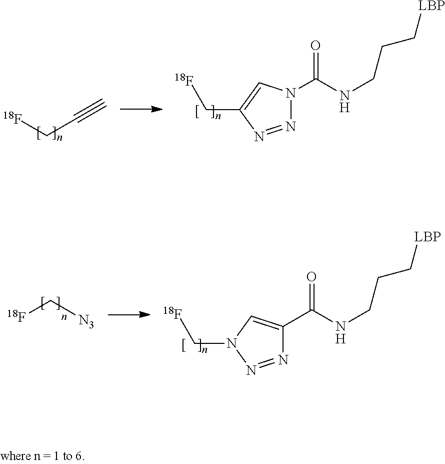

In the above reaction scheme, n is preferably 1 to 3.

In another embodiment, most preferred substituents of Formula X comprise organosilicon derivatives having ¹⁸F—Si bonds:

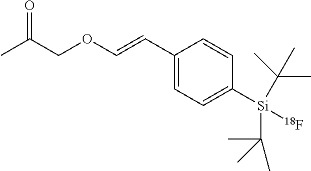 [SMILES: CC(=O)CO/C=C/c1ccc([Si]([18F])(C(C)(C)C)C(C)(C)C)cc1]

When Z¹ is ¹⁸F coordinated to the metal of a metal complex, a preferred metal is aluminium. The aluminium is preferably a metal complex of an aminocarboxylate ligand. The term “aminocarboxylate ligand” has its conventional meaning, and refers to a chelating agent where the donor atoms are a mixture of amine (N) donors and carboxylic acid (O) donors. Such chelators may be open chain (e.g. EDTA, DTPA or HBED), or macrocyclic (eg. DOTA or NOTA). Suitable such chelators include DOTA, HBED and NOTA, which are well known in the art. A preferred such chelator for aluminium is NOTA.

Preferably, the imaging agent is provided in sterile form, i.e. in a form suitable for mammalian administration as is described in the fourth aspect (below).

The imaging agents of the first aspect can be obtained as described in the third aspect (below).

In a second aspect, the present invention provides a precursor of Formula III:

Z³-(L)ₙ-[LBP]-Z² (III)
wherein:
L, n, LBP and Z² are as defined in the first aspect;
Z³ is a functional group which is chosen from:
(i) an amino-oxy group;
(ii) an azide group;
(iii) an alkyne group;
(iv) a nitrile oxide;
(iv) an aluminium, indium or gallium metal complex of an aminocarboxylate ligand.

Preferred aspects of L, n, LBP, Z² and the metal complex in the second aspect are as defined in the first aspect (above).

By the term “amino-oxy group” is meant the LBP peptide of Formula III having covalently conjugated thereto an amino-oxy functional group. Such groups are of formula —O—NH₂, preferably —CH₂O—NH₂ and have the advantage that the amine of the amino-oxy group is more reactive than a Lys amine group in condensation reactions with aldehydes to form oxime ethers. Such amino-oxy groups are suitably attached at the Cys or Lys residue of the LBP.

The precursor of the second aspect is non-radioactive. Preferably, the precursor is provided in sterile form, to facilitate the preparation of imaging agents in pharmaceutical composition form—as is described in the fourth aspect (below).

In Formula III, Z³ is preferably attached to Cys^{a} and optionally also Xaa of LBP. Preferably, Z³ is attached only to Cys^{a} of the LBP.

Amino-oxy functionalised LBP peptides can be prepared by the methods of Poethko et al [J. Nucl. Med., 45, 892-902 (2004)], Schirrmacher et al [Bioconj. Chem., 18, 2085-2089 (2007)], Solbakken et al [Bioorg. Med. Chem. Lett, 16, 6190-6193 (2006)] or Glaser et al [Bioconj. Chem., 19, 951-957 (2008)]. The amino-oxy group may optionally be conjugated in two steps. First, the N-protected amino-oxy carboxylic acid or N-protected amino-oxy activated ester is conjugated to the LBP peptide. Second, the intermediate N-protected amino-oxy functionalised LBP peptide is deprotected to give the desired product [see Solbakken and Glaser papers cited above]. N-protected amino-oxy carboxylic acids such as Boc-NH—O—CH₂(C═O)OH and Eei-N—O—CH₂(C═O)OH are commercially available, e.g. from Novabiochem and IRIS. The term “protected” refers to the use of a protecting group. By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Amine protecting groups are well known to those skilled in the art and are suitably chosen from: Boc (where Boc is tert-butyloxycarbonyl); Eei (where Eei is ethoxyethylidene); Fmoc (where Fmoc is fluorenylmethoxycarbonyl); trifluoroacetyl; allyloxycarbonyl; Dde [i.e. 1-(4,4-dimethyl-2,6-dioxocyclohexylidene)ethyl] or Npys (i.e. 3-nitro-2-pyridine sulfenyl). The use of further protecting groups are described in ‘Protective Groups in Organic Synthesis’, 4^{th} Edition, Theorodora W. Greene and Peter G. M. Wuts, [Wiley Blackwell, (2006)]. Preferred amine protecting groups are Boc and Eei, most preferably Eei.

Methods of functionalising peptides with azide groups are described by Nwe et al [Cancer Biother. Radiopharm., 24(3), 289-302 (2009)]. Li et al provide the synthesis of a compound of the type N₃-L¹-CO₂H, where L¹ is —(CH₂)₄— and its use to conjugate to amine-containing biomolecules [Bioconj. Chem., 18(6), 1987-1994 (2007)]. Hausner et al describe related methodology for N₃-L¹-CO₂H, where L¹ is —(CH₂)₂-[J. Med. Chem., 51(19), 5901-5904 (2008)]. De Graaf et al [Bioconj. Chem., 20(7), 1281-1295 (2009)] describe non-natural amino acids having azide side chains and their site-specific incorporation in peptides or proteins for subsequent click conjugation.

Methods of functionalising peptides with alkyne groups are described by Nwe et al [Cancer Biother. Radiopharm., 24(3), 289-302 (2009)]. Smith et al provide the synthesis of alkyne-functionalised isatin precursors, where the isatin compound is specific for caspase-3 or caspase-7 [J. Med. Chem., 51(24), 8057-8067 (2008)]. De Graaf et al [Bioconj. Chem., 20(7), 1281-1295 (2009)] describe non-natural amino acids having alkyne side chains and their site-specific incorporation in peptides or proteins for subsequent click conjugation.

The term “nitrile oxide” refers to a substituent of formula —C≡N⁺—O⁻. Click cycloaddition with ¹⁸F-labelled alkynes, under the conditions described above, leads to isoxazole rings. The nitrile oxides can be obtained by the methods described by Ku et al [Org. Lett., 3(26), 4185-4187 (2001)], and references therein. Thus, they are typically generated in situ by treatment of an alpha-halo aldoxime with an organic base such as triethylamine. A preferred method of generation, as well as conditions for the subsequent click cyclisation to the desired isoxazole are described by Hansen et al [J. Org. Chem., 70(19), 7761-7764 (2005)]. Hansen et al generate the desired alpha-halo aldoxime in situ by reaction of the corresponding aldehyde with chloramine-T trihydrate. See also K. B. G. Torsell “Nitrile Oxides, Nitrones and Nitronates in Organic Synthesis” [VCH, New York (1988)].

Methods of preparing functionalised NOTA chelators, their conjugation with peptides and the radiolabelling of the chelator conjugates with ¹⁸F are described by McBride et at [J. Nucl. Med., 51(3), 454-461 (2009); Bioconj. Chem., 21(7), 1331-1340 (2010)], and Layerman et al [J. Nucl. Med., 51(3), 454-461 (2010)].

In a third aspect, the present invention provides a method of preparation of the imaging agent of the first aspect, which comprises reaction of either the precursor of the second aspect or the LBP peptide as described in the first aspect, with a supply of ¹⁸F in suitable chemical form, in a suitable solvent.

Preferred aspects of the precursor and the LBP peptide in the third aspect are each as described in the first and second aspects of the present invention (above).

The “suitable solvent” is typically aqueous in nature, and is preferably a biocompatible carrier solvent as defined in the fourth aspect (below).

The “supply of ¹⁸F in suitable chemical form” is chosen depending on the functional group of the precursor or LBP peptide. When an amine group of a Lys residue or the amino group of Cys^{a} of the LBP peptide is used, then the chemical form of the ¹⁸F is suitably an active ester or an ¹⁸F-labelled carboxylic acid in the presence of an activating agent. By the term “activating agent” is meant a reagent used to facilitate coupling between an amine and a carboxylic acid to generate an amide. Suitable such activating agents are known in the art and include carbodiimides such as EDC[N-(3-dimethylaminopropyl)-N′-ethylcarbodiimide and N,N′-dialkylcarbodiimides such as dicyclohexylcarbodiimide or diisopropylcarbodiimide; and triazoles such as HBTU [O-(benzotriazol-1-yl)-N,N,N′,N′-tetramethyluronium hexafluorophosphate], HATU [O-(7-azabenzotriazol-1-yl)-N,N,N′,N′-tetramethyluronium hexafluorophosphate], and PyBOP [benzotriazol-1-yloxy)tripyrrolidinophosphonium hexafluorophosphate]. Such activating agents are commercially available. Further details are given in “March's Advanced Organic Chemistry”, 5^{th} Edition, pages 508-510, Wiley Interscience (2001). A preferred such activating agent is EDC.

¹⁸F-labelled activated esters, such as [¹⁸F]SFB can be prepared by the method of Glaser et al, and references therein [J. Lab. Comp. Radiopharm., 52, 327-330 (2009)], or the automated method of Marik et al [Appl. Rad. Isot., 65(2), 199-203 (2007)]:

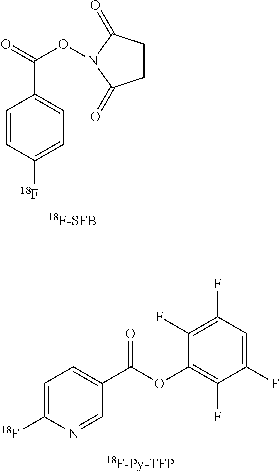

Olberg et al [J. Med. Chem., 53(4), 1732-1740 (2010)] have reported that ¹⁸F-Py-TFP (the tetrafluorophenyl ester of fluoronicotinic acid), has advantages over ¹⁸F—SFB for ¹⁸F-labelling of peptides.

¹⁸F-labelled carboxylic acids can be obtained by the method of Marik et al cited above.

When the precursor comprises an amino-oxy group, the suitable chemical form is an ¹⁸F-fluorinated aldehyde, preferably ¹⁸F-fluorobenzaldehyde or p-(di-tert-butyl-¹⁸F-fluorosilyl)benzaldehyde (¹⁸F—SiFA-A), more preferably ¹⁸F-fluorobenzaldehyde. ¹⁸F-labelled aliphatic aldehydes of formula ¹⁸F(CH₂)₂O[CH₂CH₂O]_{q}CH₂CHO, where q is 3, can be obtained by the method of Glaser et al [Bioconj. Chem., 19(4), 951-957 (2008)]. ¹⁸F-fluorobenzaldehyde can be obtained by the method of Glaser et al [J. Lab. Comp. Radiopharm., 52, 327-330 (2009)]. The precursor to ¹⁸F-fluorobenzaldehyde, i.e. Me₃N⁺—C₆H₄—CHO. CF₃SO₃⁻ is obtained by the method of Haka et al [J. Lab. Comp. Radiopharm., 27, 823-833 (1989)].

¹⁸F—SiFA-A, i.e. ¹⁸F—Si(Bu^{t})₂—C₆H₄—CHO can be obtained by the method of Schirrmacher et al [Ang. Chem. Int. Ed. Engl., 45(36), 6047-6050 (2006); Bioconj. Chem., 18(6), 2085-2089 (2007) and Bioconj. Chem., 20(2), 317-321 (2009)]. Schirrmacher et al also disclose methods of ¹⁸F-radiolabelling of amino-oxy functionalised peptides precursors using ¹⁸F—SiFA-A.

When the precursor comprises an azide-functionalised LBP peptide, the suitable chemical form is an ¹⁸F-labelled terminal alkyne. Such radiofluorinated alkynes can be obtained by the method of Kim et al [Appl. Rad. Isotop., 68(2), 329-333 (2010)], or Marik et al [Tet. Lett., 47, 6681-6684 (2006)].

When the precursor comprises an alkyne-functionalised LBP peptide, the suitable chemical form is an ¹⁸F-labelled terminal azide. A preferred such compound is ¹⁸F-fluoroethyl azide as described by Gaeta et al [Bioorg. Med. Chem. Lett., 20(15), 4649-4652 (2010)] and Glaser et al [Bioconj. Chem., 18(3), 989-993 (2007)].

When the precursor comprises an alkyne-functionalised or azide-functionalised LBP peptide, the radiofluorination reaction involves click chemistry. A suitable solvent for such click reactions is, for example acetonitrile, a C₁₋₄ alkylalcohol, dimethylformamide, tetrahydrofuran, or dimethylsulfoxide, or aqueous mixtures of any thereof, or water. Aqueous buffers can be used in the pH range of 4-8, more preferably 5-7. The reaction temperature is preferably 5 to 100° C., more preferably at 75 to 85° C., most preferably at ambient temperature (typically 15-37° C.). The click cycloaddition may optionally be carried out in the presence of an organic base, as is described by Meldal and Tornoe [Chem. Rev. 108 (2008) 2952, Table 1 (2008)].

The click reactions are carried out in the presence of a click cycloaddition catalyst. By the term “click cycloaddition catalyst” is meant a catalyst known to catalyse the click (alkyne plus azide) or click (alkyne plus isonitrile oxide) cycloaddition reaction, giving triazole and isoxazole rings respectively. Suitable such catalysts are known in the art for use in click cycloaddition reactions. Preferred such catalysts include Cu(I), and are described below. Further details of suitable catalysts are described by Wu and Fokin [Aldrichim. Acta, 40(1), 7-17 (2007)] and Meldal and Tornoe [Chem. Rev., 108, 2952-3015 (2008)].

A preferred click cycloaddition catalyst comprises Cu(I). The Cu(I) catalyst is present in an amount sufficient for the reaction to progress, typically either in a catalytic amount or in excess, such as 0.02 to 1.5 molar equivalents relative to the azide or isonitrile oxide reactant. Suitable Cu(I) catalysts include Cu(I) salts such as CuI or [Cu(NCCH₃)₄][PF₆], but advantageously Cu(II) salts such as copper (II) sulphate may be used in the presence of a reducing agent to generate Cu(I) in situ. Suitable reducing agents include: ascorbic acid or a salt thereof for example sodium ascorbate, hydroquinone, metallic copper, glutathione, cysteine, Fe²⁺, or Co²⁺. Cu(I) is also intrinsically present on the surface of elemental copper particles, thus elemental copper, for example in the form of powder or granules may also be used as catalyst. Elemental copper, with a controlled particle size is a preferred source of the Cu(I) catalyst. A more preferred such catalyst is elemental copper as copper powder, having a particle size in the range 0.001 to 1 mm, preferably 0.1 mm to 0.7 mm, more preferably around 0.4 mm. Alternatively, coiled copper wire can be used with a diameter in the range of 0.01 to 1.0 mm, preferably 0.05 to 0.5 mm, and more preferably with a diameter of 0.1 mm. The Cu(I) catalyst may optionally be used in the presence of bathophenanthroline, which is used to stabilise Cu(I) in click chemistry.

Further details of ¹⁸F-labelling of peptides using click, active ester and metal complex methodology are provided by Olberg et al [J. Med. Chem., 53(4), 1732-1740 (2010) and Curr. Top. Med. Chem., 10(16), 1669-1679 (2010)].

Certain LBP peptides are commercially available. Thus, cinnamycin and duramycin are available from Sigma-Aldrich. Duramycin is produced by the strain: D3168 Duramycin from Streptoverticillium cinnamoneus. Cinnamycin can be biochemically produced by several strains, eg. from Streptomyces cinnamoneus or from Streptoverticillium griseoverticillatum. See the review by C. Chatterjee et al [Chem. Rev., 105, 633-683 (2005)].

Other peptides can be obtained by solid phase peptide synthesis as described in P. Lloyd-Williams, F. Albericio and E. Girald; Chemical Approaches to the Synthesis of Peptides and Proteins, CRC Press, 1997.

In a fourth aspect, the present invention provides a radiopharmaceutical composition which comprises the imaging agent of the first aspect, together with a biocompatible carrier, in a form suitable for mammalian administration.

Preferred aspects of the imaging agent in the fourth aspect are as described in the first aspect of the present invention (above).

By the phrase “in a form suitable for mammalian administration” is meant a composition which is sterile, pyrogen-free, lacks compounds which produce toxic or adverse effects, and is formulated at a biocompatible pH (approximately pH 4.0 to 10.5). Such compositions lack particulates which could risk causing emboli in vivo, and are formulated so that precipitation does not occur on contact with biological fluids (e.g. blood). Such compositions also contain only biologically compatible excipients, and are preferably isotonic.

The “biocompatible carrier” is a fluid, especially a liquid, in which the imaging agent can be suspended or preferably dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous buffer solution comprising a biocompatible buffering agent (e.g. phosphate buffer); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). Preferably the biocompatible carrier is pyrogen-free water for injection, isotonic saline or phosphate buffer.

The imaging agents and biocompatible carrier are each supplied in suitable vials or vessels which comprise a sealed container which permits maintenance of sterile integrity and/or radioactive safety, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour.

Preferred multiple dose containers comprise a single bulk vial (e.g. of 10 to 50 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pharmaceutical compositions of the present invention preferably have a dosage suitable for a single patient and are provided in a suitable syringe or container, as described above.

The pharmaceutical composition may contain additional optional excipients such as: an antimicrobial preservative, pH-adjusting agent, filler, radioprotectant, solubiliser or osmolality adjusting agent. By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation as described above. By the term “solubiliser” is meant an additive present in the composition which increases the solubility of the imaging agent in the solvent. A preferred such solvent is aqueous media, and hence the solubiliser preferably improves solubility in water. Suitable such solubilisers include: C₁₋₄ alcohols; glycerine; polyethylene glycol (PEG); propylene glycol; polyoxyethylene sorbitan monooleate; sorbitan monooloeate; polysorbates; poly(oxyethylene)poly(oxypropylene)poly(oxyethylene) block copolymers (Pluronics™); cyclodextrins (e.g. alpha, beta or gamma cyclodextrin, hydroxypropyl-β-cyclodextrin or hydroxypropyl-γ-cyclodextrin) and lecithin.

By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the composition is within acceptable limits (approximately pH 4.0 to 10.5) for human or mammalian administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The radiopharmaceutical compositions of the fourth aspect may be prepared under aseptic manufacture (i.e. clean room) conditions to give the desired sterile, non-pyrogenic product. It is preferred that the key components, especially the associated reagents plus those parts of the apparatus which come into contact with the imaging agent (eg. vials) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise some components in advance, so that the minimum number of manipulations needs to be carried out. As a precaution, however, it is preferred to include at least a sterile filtration step as the final step in the preparation of the pharmaceutical composition.

The radiopharmaceutical compositions of the present invention may be prepared by various methods:
(i) aseptic manufacture techniques in which the ¹⁸F-radiolabelling step is carried out in a clean room environment;
(ii) terminal sterilisation, in which the ¹⁸F-radiolabelling is carried out without using aseptic manufacture and then sterilised at the last step [eg. by gamma irradiation, autoclaving dry heat or chemical treatment (e.g. with ethylene oxide)];
(iii) kit methodology in which a sterile, non-radioactive kit formulation comprising a suitable precursor of Formula III and optional excipients is reacted with a suitable supply of ¹⁸F;
(iv) aseptic manufacture techniques in which the ¹⁸F-radiolabelling step is carried out using an automated synthesizer apparatus.

Method (iv) is preferred. Kits for use in this method are described in the fifth embodiment (below).

By the term “automated synthesizer” is meant an automated module based on the principle of unit operations as described by Satyamurthy et al [Clin. Positr. Imag., 2(5), 233-253 (1999)]. The term ‘unit operations’ means that complex processes are reduced to a series of simple operations or reactions, which can be applied to a range of materials. Such automated synthesizers are preferred for the method of the present invention especially when a radiopharmaceutical composition is desired. They are commercially available from a range of suppliers [Satyamurthy et al, above], including: GE Healthcare; CTI Inc; Ion Beam Applications S.A. (Chemin du Cyclotron 3, B-1348 Louvain-La-Neuve, Belgium); Raytest (Germany) and Bioscan (USA).

Commercial automated synthesizers also provide suitable containers for the liquid radioactive waste generated as a result of the radiopharmaceutical preparation. Automated synthesizers are not typically provided with radiation shielding, since they are designed to be employed in a suitably configured radioactive work cell. The radioactive work cell provides suitable radiation shielding to protect the operator from potential radiation dose, as well as ventilation to remove chemical and/or radioactive vapours. The automated synthesizer preferably comprises a cassette. By the term “cassette” is meant a piece of apparatus designed to fit removably and interchangeably onto an automated synthesizer apparatus (as defined above), in such a way that mechanical movement of moving parts of the synthesizer controls the operation of the cassette from outside the cassette, i.e. externally. Suitable cassettes comprise a linear array of valves, each linked to a port where reagents or vials can be attached, by either needle puncture of an inverted septum-sealed vial, or by gas-tight, marrying joints. Each valve has a male-female joint which interfaces with a corresponding moving arm of the automated synthesizer. External rotation of the arm thus controls the opening or closing of the valve when the cassette is attached to the automated synthesizer. Additional moving parts of the automated synthesizer are designed to clip onto syringe plunger tips, and thus raise or depress syringe barrels.

The cassette is versatile, typically having several positions where reagents can be attached, and several suitable for attachment of syringe vials of reagents or chromatography cartridges (eg. solid phase extraction or SPE). The cassette always comprises a reaction vessel. Such reaction vessels are preferably 1 to 10 cm³, most preferably 2 to 5 cm³ in volume and are configured such that 3 or more ports of the cassette are connected thereto, to permit transfer of reagents or solvents from various ports on the cassette. Preferably the cassette has 15 to 40 valves in a linear array, most preferably 20 to 30, with 25 being especially preferred. The valves of the cassette are preferably each identical, and most preferably are 3-way valves. The cassettes are designed to be suitable for radiopharmaceutical manufacture and are therefore manufactured from materials which are of pharmaceutical grade and ideally also are resistant to radiolysis.

Preferred automated synthesizers of the present invention comprise a disposable or single use cassette which comprises all the reagents, reaction vessels and apparatus necessary to carry out the preparation of a given batch of radiofluorinated radiopharmaceutical. The cassette means that the automated synthesizer has the flexibility to be capable of making a variety of different radiopharmaceuticals with minimal risk of cross-contamination, by simply changing the cassette. The cassette approach also has the advantages of: simplified set-up hence reduced risk of operator error; improved GMP (Good Manufacturing Practice) compliance; multi-tracer capability; rapid change between production runs; pre-run automated diagnostic checking of the cassette and reagents; automated barcode cross-check of chemical reagents vs the synthesis to be carried out; reagent traceability; single-use and hence no risk of cross-contamination, tamper and abuse resistance.

Included in this aspect of the invention, is the use of an automated synthesizer apparatus to prepare the radiopharmaceutical composition of the second aspect. In a fifth aspect, the present invention provides a kit for the preparation of the radiopharmaceutical composition of the fourth aspect, which comprises the precursor of the second aspect or the LBP peptide as defined in the first aspect in sterile, solid form such that upon reconstitution with a sterile supply of ¹⁸F in suitable chemical form, dissolution occurs to give the desired radiopharmaceutical composition.

The term “suitable chemical form” is as defined in the third aspect (above).

Preferred aspects of the precursor in the fifth aspect are as described in the second aspect of the present invention (above).

By the term “kit” is meant one or more non-radioactive pharmaceutical grade containers, comprising the necessary chemicals to prepare the desired radiopharmaceutical composition, together with operating instructions. The kit is designed to be reconstituted with ¹⁸F to give a solution suitable for human administration with the minimum of manipulation.

The sterile, solid form is preferably a lyophilised solid.

The non-radioactive kits may optionally further comprise additional components such as a transchelator, radioprotectant, antimicrobial preservative, pH-adjusting agent or filler—as defined above.

Included in this aspect of the invention, is the use of a cassette which comprises the kit of the fifth aspect in conjunction with an automated synthesizer apparatus to prepare the radiopharmaceutical composition of the second aspect.

In a sixth aspect, the present invention provides a method of imaging the human or animal body which comprises generating an image of at least a part of said body to which the imaging agent of the first aspect, or the composition of the fourth aspect has distributed using PET, wherein said imaging agent or composition has been previously administered to said body.

Preferred aspects of the imaging agent or composition in the sixth aspect are as described in the first and fourth aspects respectively of the present invention (above). The method of the sixth aspect is preferably carried out where the part of the body is disease state where abnormal apoptosis is involved. By the term “abnormal apoptosis” is meant dysregulation of the programmed cell death process. Such dysregulation has been implicated in a number of disease states, including those associated with the inhibition of apoptosis, such as cancer and autoimmune disorders, and those associated with hyperactive apoptosis, including neurodegenerative diseases, haematologic diseases, AIDS, ischaemia and allograft rejection.

There is also emerging evidence that apoptosis contributes to the instability of the atherosclerotic lesions. Plaques vulnerable to rupture typically have a large necrotic core and an attenuated fibrous cap, which is significantly infiltrated by macrophages and lymphocytes. Although the consequences of cell death within the advance lesion are not precisely defined, morphological data suggest that apoptosis of macrophages contributes substantially to the size of the necrotic core, whereas apoptosis of smooth muscle cells (SMCs) results in thinning of the fibrous cap. Extensive apoptosis of macrophages is believed to occur at sites of plaque rupture, and possibly contributes to the process of rupture. Therefore, detection of apoptosis may help identify atherosclerotic lesions prone to rupture.

The visualization and quantitation of apoptosis is therefore useful in the diagnosis of such apoptosis-related pathophysiology.

The imaging method of the sixth aspect may optionally be carried out repeatedly to monitor the effect of treatment of a human or animal body with a drug, said imaging being effected before and after treatment with said drug, and optionally also during treatment with said drug. Therapeutic treatments for these diseases aim to restore balanced apoptosis, either by stimulating or inhibiting the PCD process as appropriate. Of particular interest is early monitoring of the efficacy of cancer therapy to ensure that malignant growth is controlled before the condition becomes terminal.

In a seventh aspect, the present invention provides the use of the imaging agent of the first aspect, the composition of the fourth aspect, or the kit of the fifth aspect in a method of diagnosis of the human or animal body.

Preferred aspects of the imaging agent or composition in the seventh aspect are as described in the first and fourth aspects respectively of the present invention (above). The use of the seventh aspect is preferably where the diagnosis of the human or animal body is of a disease state where abnormal apoptosis is involved. Such “abnormal apoptosis” is as described in the sixth aspect (above).

The invention is illustrated by the non-limiting Examples detailed below. Example 1 and Example 2 provide the syntheses of Precursor 1A and Precursor 1B respectively, amino-oxy functionalised LBP peptides of the invention protected with two different amino-protecting groups. Example 3 provides the synthesis of Precursor 2, an amino-oxy functionalised LBP peptides of the invention. Example 4 provides the synthesis of Compound 1, a non-radioactive fluorinated compound of the invention where the fluorine isotope is ¹⁹F. Compound 1 is useful for determining biological binding properties of the ¹⁸F counterpart (Compound 1A). Example 5 provides a method of ¹⁸F-labelling Precursor 1 using ¹⁸F-benzaldehyde, to give an ¹⁸F-labelled compound of the invention (Compound 1A). Example 6 provides binding affinity data for phosphatidylethanolamine and demonstrates that the generation of Compound 1 has no significant effect on the binding affinity. Compound 1A was assessed by biodistribution in the EL4 mouse lymphoma xenograft model. The results from this work is provided in Example 7.

### ABBREVIATIONS

Conventional single letter or 3-letter amino acid abbreviations are used.

### Ac: Acetyl.

### ACN: Acetonitrile.

Boc: tert-Butyloxycarbonyl.
DIPEA: N,N□□-diisopropylethylamine.

### DMSO: Dimethylsulfoxide.

EOS: End of synthesis.

### Fmoc: 9-Fluorenylmethoxycarbonyl.

HATU: O-(7-Azabenzotriazol-1-yl)-N,N,N′,N′-tetramethyluronium hexafluorophosphate.
HPLC: High performance liquid chromatography.
NMP: 1-Methyl-2-pyrrolidinone.
PBS: Phosphate-buffered saline.
PyBOP: Benzotriazol-1-yl-oxytripyrrolidinophosphonium hexafluorophosphate.
RAC: radioactive concentration.
RCP: Radiochemical purity.
tBu: tent-Butyl.
TFA: Trifluoroacetic acid.

### TFP: Tetrafluorophenyl.

T_{R}: retention time.

TABLE 1
Compounds of the Invention.
Formula II (with bridges as specified in the first aspect):
Cys^{a}-Xaa-Gln-Ser^{b}-Cys^{c}-Ser^{d}-Phe-Gly-Pro-Phe-
Thr^{c}-Phe-Val-Cys^{b}-(HO-Asp)-Gly-Asn-Thr^{a}-Lys^{d}

| Name | Structure |
|---|---|
| LBP1 = duramycin | Formula II, where Xaa = Lys. |
| LBP2 = cinnamycin | Formula II, where Xaa = Arg. |
| Precursor 1A | [LBP1]-(CO)CH₂ONH(CO)OBu^{t} |
| | (Mixture of isomers LBP1 functionalized at either |
| | Cys^{a} or Xaa Lys groups). |
| Precursor 1B | [LBP1]-(CO)CH₂ONC(CH₃)OEt |
| | (Mixture of isomers LBP1 functionalized at either |
| | Cys^{a} or Xaa Lys groups). |
| Precursor 2 | [LBP1]-(CO)CH₂ONH₂ |
| | (Mixture of isomers LBP1 functionalized at either |
| | Cys^{a} or Xaa Lys groups). |
| Compound 1 | [LBP1]-(CO)CH₂O—N═CH—C₆H₄—F |
| | (Mixture of isomers LBP1 functionalized at either |
| | Cys^{a} or Xaa Lys groups). |
| Compound 1A | [LBP1]-(CO)CH₂O—N═CH—C₆H₄—¹⁸F |
| | (Mixture of isomers LBP1 functionalized at either |
| | Cys^{a} or Xaa Lys groups). |

### Example 1

### Synthesis of (Boc-aminooxy)acetyl-Duramycin (Precursor 1A)

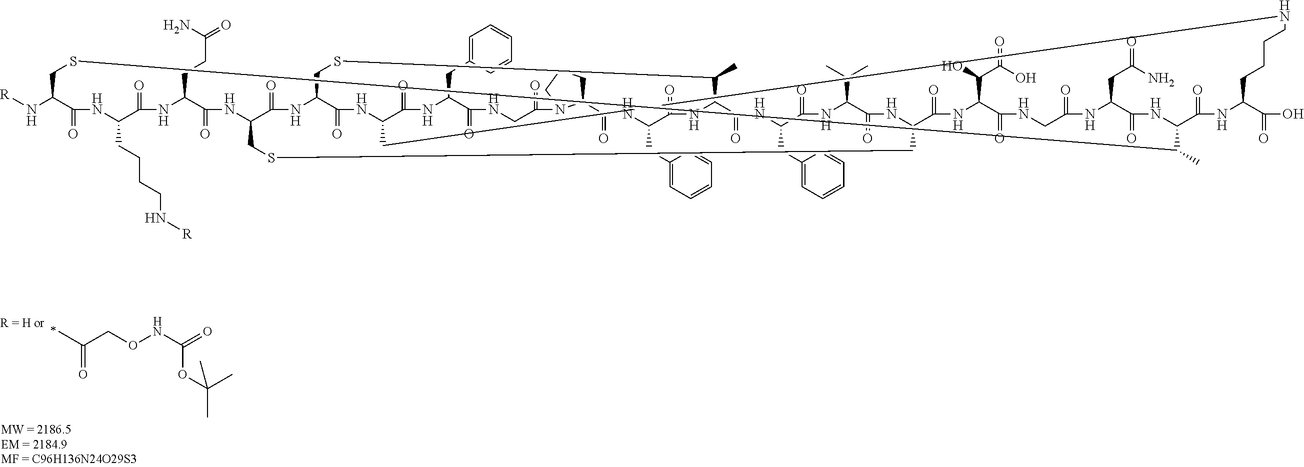

Duramycin (Sigma-Aldrich; 8.0 mg, 4.0 μmol), (Boc-aminooxy)acetic acid TFP ester (Invitrogen; 1.3 mg, 3.8 μmol) and DIPEA (2.1 μL, 12.5 μmol) were dissolved in NMP (1 mL). The reaction mixture was shaken for 30 min. The mixture was then diluted with water/0.1% TFA (6 mL) and the product purified using preparative HPLC.

Purification was by preparative HPLC (Beckman System Gold chromatography system using the following conditions: solvent A=H₂O/0.1% TFA and solvent B=ACN/0.1% TFA, gradient: 20-50% B over 40 min; flow rate: 10 mL/min; column: Phenomenex Luna 5 μm C18 (2) 250×21.2 mm; detection: UV 214 nm), afforded 3.8 mg pure Precursor 1A (yield 44%). The purified material was analysed by analytical LC-MS (gradient: 20-70% B over 5 min, t_{R}: 1.93 min, found m/z: 1093.7, expected MH₂²⁺: 1093.5).

Separation of the Precursor 1 regioisomers could not be achieved under the above analytical or preparative HPLC conditions. In each case the two regioisomers eluted as a single peak.

Separation of the Precursor 1A regioisomers can, however, be achieved by analytical HPLC under more gentle eluting conditions: LC-MS gradient 25-35% B over 5 min, t_{R}: 2.0 min, found m/z: 1093.7 and t_{R}: 2.3 min, found m/z: 1093.7, expected MH₂²⁺: 1093.5. Similar conditions can be used by preparative HPLC to isolate each regioisomer.

### Example 2

### Synthesis of (Eei-aminooxy)acetyl-Duramycin (Precursor 1B)

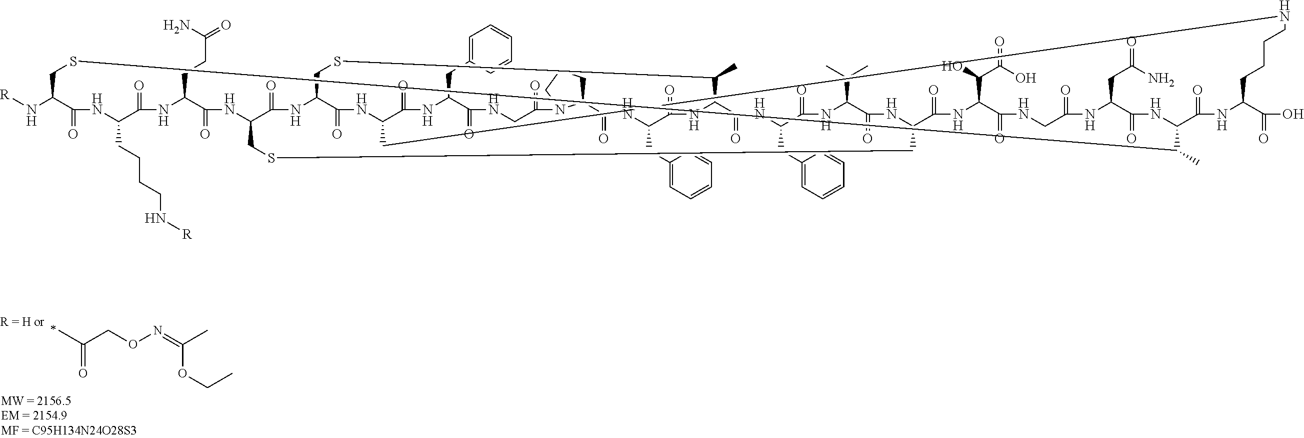

Duramycin (Sigma-Aldrich; 50 mg, 25 μmol), (Eei-aminooxy)acetic acid NHS ester (Iris Biotech., 5.1 mg, 20 μmol) and DIPEA (17 μL, 100 μmol) were dissolved in NMP (1 mL). The reaction mixture was shaken for 45 min. The mixture was then diluted with water/0.1% acetic acid (8 mL) and the product purified using preparative HPLC

Purification by preparative HPLC (as for Example 1 with gradient 14-45% B over 40 min where A=water/0.1% acetic acid and B=ACN) afforded 14 mg pure Precursor 1B (yield 26%). The purified material was analysed by LC-MS (gradient: 20-50% B over 5 min, t_{R}: 2.5 and 2.7 min, found m/z: 1078.8, expected MH₂²⁺: 1078.5).

Chromatographic resolution of the (Eei-aminooxy)acetyl-Duramycin regioisomers could be achieved on analytical HPLC using 0.1% TFA. However, the Eei protecting group is labile in 0.1% TFA so preparative separation was not feasible. The regioisomers were not resolved using 0.1% acetic acid.

### Example 3

### Synthesis of Aminooxyacetyl-Duramycin (Precursor 2)

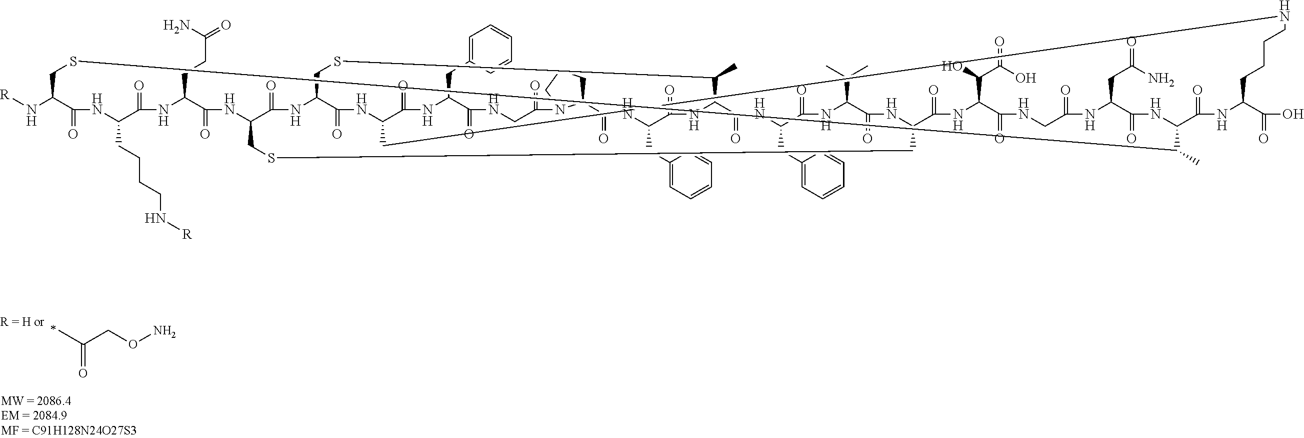

Precursor 1B (14 mg) was treated with 2.5% TFA/water (2.8 mL) under argon for 40 min. The reaction mixture was diluted with water (31 mL) and the product lyophilized (frozen under argon using isopropanol/dry-ice) affording 18 mg Precursor 2. The lyophilized product was analysed by LC-MS (gradient: 20-50% B over 5 min, t_{R}: 2.5 and 2.1 min, found m/z: 1043.8, expected MH₂²⁺: 1043.5).

Chromatographic resolution of the Precursor 2 regioisomers could be achieved on analytical HPLC using 0.1% TFA. However, due to the high reactivity of the free aminooxy group towards traces of ketones and aldehydes in the solvent and the atmosphere. no attempt was made to separate the regioisomers at this stage.

### Example 4

### Synthesis of N-(4-Fluorobenzylidene)-aminooxyacetyl-Duramycin (Compound 1)

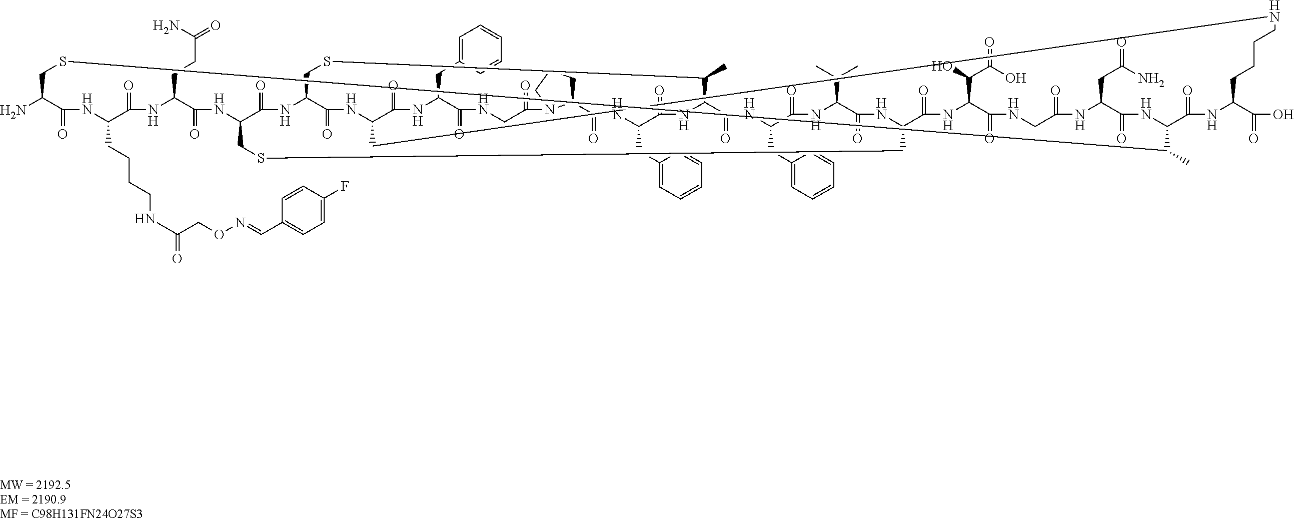 [SMILES: CC(C)[C@@H]1NC(=O)[C@H](Cc2ccccc2)NC(=O)[C@H]2NC(=O)[C@H](Cc3ccccc3)NC(=O)[C@@H]3CCCN3C(=O)CNC(=O)[C@H](Cc3ccccc3)NC(=O)[C@@H]3CNCCCC[C@@H](C(=O)O)NC(=O)[C@H]4NC(=O)[C@H](CC(N)=O)NC(=O)CNC(=O)[C@H]([C@@H](O)C(=O)O)NC(=O)[C@H](CSC[C@@H](NC(=O)[C@H](CCC(N)=O)NC(=O)[C@H](CCCCNC(=O)CO/N=C/c5ccc(F)cc5)NC(=O)[C@@H](N)CS[C@H]4C)C(=O)N[C@@H](CS[C@H]2C)C(=O)N3)NC1=O]

Precursor 1A (Example 1; 1.0 mg, 0.46 μmol) was treated with TFA (1 mL) for 30 min. The TFA was removed in vacuo and the residue redissolved in 40% ACN/water (1 mL). 4-Fluorobenzaldehyde (1.0 μl, 9.2 μmol) was added and the reaction mixture shaken for 30 min. The reaction mixture was then diluted with 20% ACN/water/0.1% TFA (6 mL) and the product purified by preparative HPLC.

Purification by preparative HPLC (as for Example 1 with gradient: 20-50% B over 40 min) afforded 0.6 mg pure Compound 1 (yield 60%). The purified material was analysed by analytical LC-MS (gradient: 20-70% B over 5 min, t_{R}: 2.09 min, found m/z: 1096.5, expected MH₂²⁺: 1096.5). Separation of the Compound 1 regioisomers could not be achieved using either analytical or preparative HPLC. In each case the two regioisomers eluted as a single peak.

### Example 5

### Radiosynthesis of Compound 1A from Precursor 2

Compound 1A is produced in a two-step procedure using an automated synthesizer and cassette (FASTlab™, GE Healthcare).

Step (a) synthesis and purification of ¹⁸F-benzaldehyde.

[¹⁸F]fluoride was produced using a GEMS PETtrace cyclotron with a silver target via the [¹⁸O](p,n) [¹⁸F] nuclear reaction. Total target volumes of 1.5-3.5 mL were used. The radiofluoride was trapped on a Waters QMA cartridge (pre-conditioned with carbonate), and the fluoride is eluted with a solution of Kryptofix_{2.2.2}. (4 mg, 10.7 μM) and potassium carbonate (0.56 mg, 4.1 μM) in water (80 μL) and acetonitrile (320 μL). Nitrogen was used to drive the solution off the QMA cartridge to the reaction vessel. The [¹⁸F]fluoride was dried for 9 minutes at 120° C. under a steady stream of nitrogen and vacuum. Trimethylammonium benzaldehyde triflate, [Haka et al, J. Lab. Comp. Radiopharm., 27, 823-833 (1989)] (3.3 mg, 10.5 μM), in dimethylsulfoxide (1.1 mL) was added to the dried [¹⁸F]fluoride, and the mixture heated at 105° C. for 7 minutes to produce 4-[¹⁸F]fluorobenzaldehyde.

The crude labelling mixture was then diluted with ammonium hydroxide solution and loaded onto an MCX+SPE cartridge (pre-conditioned with water as part of the FASTlab sequence). The cartridge was washed with water, dried with nitrogen gas before elution of 4-[¹⁸F]fluorobenzaldehyde back to the reaction vessel in ethanol (1 mL). 4-7% (decay corrected) of [¹⁸F]fluorobenzaldehyde remained trapped on the cartridge.

Step (b): Aldehyde Condensation with Amino-oxy Derivative (Precursor 2).

Precursor 2 (5 mg) was transferred to the FASTlab reaction vessel prior to elution of 4-[¹⁸F]fluorobenzaldehyde from the MCX+cartridge. The mixture was then heated at 60° C. for 5 minutes. The crude reaction material was then diluted with water and loaded onto a tC2 SPE cartridge. This was then dried with nitrogen and vacuum, washed with an ethanolic solution and dried again. Compound 1A was then eluted into a collection vial with ethanol followed by water (6 mL total). The EOS yield was 16-34% (non-decay corrected). Analytical HPLC confirmed that Compound 1A was prepared with an RCP of 97% and was stable for at least 180 min (RCP 94%, RAC 150 MBq/mL).

HPLC Conditions

Column: Phenomenex, Jupiter 4u, Proteo 90A, 250×4.6 mm.

Gradient: 0 min 50% B
5 min 50% B
20 min 90% B
25 min 90% B

Flow rate: 1 mL/min

UV detection: 254 nm.

Mobile phase A: 50 mM ammonium acetate

Mobile phase B: methanol.
Compound 1A (T_{R})=22.6 min.

### Example 6

### Affinity for Phosphatidylethanolamine

A Biacore 3000 (GE Healthcare, Uppsala) was equipped with an L1 chip. Liposomes made of POPE/POPC (20% PE) were applied for the affinity study using the capture technique recommended by the manufacturer. Each run consisted of activation of the chip surface, immobilization of liposomes, binding of peptide and wash off of both liposomes and peptide (regeneration). Similar applications can be found in Frostell-Karlsson et al [Pharm. Sciences, V.94 (1), (2005)]. Thorough washing of needle, tubing and liquid handling system with running buffer was performed after each cycle.

BIACORE software: The BIACORE control software including all method instructions was applied. A method with commands was also written in the BIACORE Method Definition Language (MDL) to have full control over pre-programmed instructions. BIACORE evaluation software was applied for analysing the sensorgrams.

Compound 1 was found to be a good binder to phosphatidyl ethanolamine. The K_{D} for duramycin and Compound 1 was both less than 100 nM. The results are given in Table 2:

TABLE 2

| | Duramycin | Compound 1 |
|---|---|---|
| k_{d} (1/s) | ~8 · 10⁻⁵ | ~12 · 10⁻⁴ |
| kₐ (1/Ms) | ~2 · 10⁴ | ~2.8 · 10⁴ |
| K_{D} (nM) | ~5 | ~43 |

### Example 7

### Tumour Uptake Studies

Compound 1A was assessed by biodistribution in the EL4 mouse lymphoma xenograft model. Briefly, following establishment of tumour growth in C57/B16 mice, the animals were treated with either:
(i) a saline/DMSO solution; or
(ii) with chemotherapy (67 mg/kg etoposide and 100 mg/kg cyclophosphamide in 50% saline 50% DMSO).

Twenty four hours after therapy or vehicle treatment, the animals were assessed for the biodistribution of Compound 1A. In addition, the tumours were extracted and assessed for levels of apoptosis by measuring caspase activity (capase-Glo assay). An increase of tumour retention of Compound 1A was observed which followed an increase in tumour apoptosis.

What is claimed is:

1. An imaging agent which comprises a compound of Formula I:
Z¹-(L)ₙ-[LBP]-Z² (I)
wherein:
LBP is a lantibiotic peptide of Formula II:
Cys^{a}-Xaa-Gln-Ser^{b}-Cys^{c}-Ser^{d}-Phe-Gly-Pro-Phe-Thr^{c}-Phe-Val-Cys^{b}-(HO-Asp)-Gly-Asn-Thr^{a}-Lys^{d} (II)
Xaa is Arg or Lys;
Cys^{a}-Thr^{a}, Ser^{b}-Cys^{b} and Cys^{c}-Thr^{c} are covalently linked via thioether bonds;
Ser^{d}-Lys^{d} are covalently linked via a lysinoalanine bond;
HO-Asp is β-hydroxyaspartic acid;
Z¹-(L)ₙ- is attached to Cys^{a} and optionally also Xaa of LBP, wherein Z¹ is either ¹⁸F or ¹⁸F coordinated to the metal of a metal complex;
Z² is attached to the C-terminus of LBP and is OH or OB^{c},
where B^{c} is a biocompatible cation; and
L is a synthetic linker group of formula -(A)ₘ- wherein each A is independently —CR₂—, —CR═CR—, —C≡C—, —CR₂CO₂—, —CO₂CR₂—, —NRCO—, —CONR—, —CR═N—O—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, —Ar—, —NR—Ar—, —O—Ar—, —Ar—(CO)—, an amino acid, a sugar or a monodisperse polyethyleneglycol (PEG) building block, wherein each Ar is independently a C₅₋₁₂ arylene group, or a C₃₋₁₂ heteroarylene group;
each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
n is an integer of value 0 or 1;

2. The imaging agent of claim 1, where Z¹ is attached only to Cys^{a} of LBP.

3. The imaging agent of claim 1, where Xaa is Arg.

4. The imaging agent of claim 1, where Z¹-(L)ₙ- comprises a group of Formula X:
¹⁸F—X¹-(A)ₓ- (X)
where: x is an integer of value 0 to 5;
X¹ is chosen from —Ar—, —Ar—NR—, —Ar—O—, —Ar—(CO)— or —Si(R^{a})₂—;
wherein A, Ar and R are as defined for the L group in claim 1, and each R^{a} is independently C₁₋₉ alkyl.

5. The imaging agent of claim 4, where Ar¹ comprises a phenyl ring or a heterocyclic ring chosen from a triazole, isoxazole or pyridine ring.

6. The imaging agent of claim 1, where Z¹ comprises an aluminium complex of an aminocarboxylate ligand, wherein the ¹⁸F radiolabel is coordinated to said aluminium of said complex.

7. A precursor of Formula III:
Z³-(L)ₙ-[LBP]-Z² (III)
wherein:
L, n, LBP and Z² are as defined in claim 1;
Z³ is a functional group which is chosen from:
(i) an amino-oxy group;
(ii) an azide group;
(iii) an alkyne group;
(iv) a nitrile oxide;
(v) an aluminium, indium or gallium metal complex of an aminocarboxylate ligand.

8. A method of preparation of the imaging agent of claim 1, which comprises reaction of the LBP peptide as defined in claim 1, with a supply of ¹⁸F in suitable chemical form, in a suitable solvent.

9. A radiopharmaceutical composition which comprises the imaging agent of claim 1, together with a biocompatible carrier, in a form suitable for mammalian administration.

10. A kit for the preparation of a radiopharmaceutical composition, which comprises the LBP peptide as defined in claim 1 in sterile, solid form such that upon reconstitution with a sterile supply of ¹⁸F in suitable chemical form, dissolution occurs to give the desired radiopharmaceutical composition.

11. The kit of claim 10, where the sterile, solid form is a lyophilised solid.

12. A method of imaging the human or animal body which comprises generating an image of at least a part of said body to which the imaging agent of claim 1 has distributed using PET, wherein said imaging agent or composition has been previously administered to said body.

13. The method of claim 12, where said part of the body is a disease state where abnormal apoptosis is involved.

14. The method of claim 12, which is carried out repeatedly to monitor the effect of treatment of a human or animal body with a drug, said imaging being effected before and after treatment with said drug, and optionally also during treatment with said drug.

15. (canceled)

16. (canceled)

17. A method of preparation of an imaging agent, which comprises reaction of the precursor of claim 8 with a supply of ¹⁸F in suitable chemical form, in a suitable solvent.

18. A kit for the preparation of a radiopharmaceutical composition, which comprises the precursor of claim 7 in sterile, solid form such that upon reconstitution with a sterile supply of ¹⁸F in suitable chemical form, dissolution occurs to give the desired radiopharmaceutical composition.

19. The kit of claim 18, where the sterile, solid form is a lyophilised solid.

20. A method of imaging the human or animal body which comprises generating an image of at least a part of said body to which the composition of claim 9 has distributed using PET, wherein said imaging agent or composition has been previously administered to said body.

21. The method of claim 20, where said part of the body is a disease state where abnormal apoptosis is involved.

22. The method of claim 20, which is carried out repeatedly to monitor the effect of treatment of a human or animal body with a drug, said imaging being effected before and after treatment with said drug, and optionally also during treatment with said drug.
